(19) United States
(12) Patent Application Publication
(10) Pub. No.: US 2018/0008341 A1
(43) Pub. Date: Jan. 11, 2018

(54) SYSTEM AND METHOD FOR DISPLAYING AN ACTIVE HEATING ZONE DURING AN ABLATION PROCEDURE

(71) Applicant: COVIDIEN LP, MANSFIELD, MA (US)

(72) Inventor: JOSEPH D. BRANNAN, LYONS, CO (US)

(21) Appl. No.: 15/202,840

(22) Filed: Jul. 6, 2016

(51) Int. Cl. A61B 18/14; A61B 18/00

(52) U.S. Cl. CPC A61B 18/1477; A61B 2018/00803; A61B 2018/00577; A61B 2018/00642; A61B 2018/00702

(57) ABSTRACT

A method of generating a representation of an active heating zone on a display in real time during an ablation procedure includes processing imaging data of a surgical site generated by an imaging device, navigating an ablation device in proximity to target tissue, delivering electrosurgical energy to the target tissue via the ablation device to generate an active heating zone, detecting a Doppler shift in the imaging data based on the delivery of electrosurgical energy to the target tissue, and generating a representation of the active heating zone relative to the surgical site based on the detected Doppler shift.

### BACKGROUND

### 1. Technical Field

The present disclosure relates to methods of ablating tissue and, more specifically, to a system and method for displaying an active heating zone during an ablation procedure.

### 2. Discussion of Related Art

Electromagnetic fields can be used to heat and destroy tumor cells. Treatment may involve inserting an ablation probe into tissue where cancerous tumors have been identified. Once the ablation probe is properly positioned, the ablation probe induces electromagnetic fields within the tissue surrounding the ablation probe to treat the tissue.

In the treatment of diseases such as cancer, certain types of tumor cells have been found to denature at elevated temperatures. In certain tissues (e.g., bone), known treatment methods heat diseased cells to temperatures above 60° C. to kill the diseased cells. During an ablation procedure, target tissue is ablated while avoiding ablation of surrounding healthy tissue. Ultrasound imaging is typically used to visualize ablated tissue after an ablation procedure to assess the effectiveness of the ablation. However, this does not allow a clinician to observe, in real time during the ablation procedure, where in a surgical site an active heating zone generated in the tissue by an ablation device will cause an ablation lesion to be formed. Additionally, ablation energy may interfere with ultrasound imaging. A system and method that displays a representation of an active heating zone generated by a treatment device within an image of a surgical site, in real time during an ablation procedure, would allow a clinician to quickly detect the position of the treatment device and to adjust the position of the treatment device within the surgical site to prevent damage to healthy tissue.

### SUMMARY

According to an embodiment of the present disclosure, a method of generating a representation of an active heating zone on a display in real time during an ablation procedure is provided. The method includes processing imaging data of a surgical site generated by an imaging device, navigating an ablation device in proximity to target tissue, delivering electrosurgical energy to the target tissue via the ablation device to generate an active heating zone, detecting a Doppler shift in the imaging data based on the delivery of electrosurgical energy to the target tissue, and generating a representation of the active heating zone relative to the surgical site based on the detected Doppler shift.

According to an aspect of the above-described embodiments, the Doppler shift associated with the active heating zone is unique to the active heating zone and may be distinguished from a Doppler shift associated with other physiological features such as blood flow, as described in detail herein.

According to an aspect of the above-described embodiment, generating the representation of the active heating zone includes indicating a clinical outcome prediction zone based on active heating intensity. The imaging device may be an ultrasound probe. The ablation device may be a microwave antenna configured to deliver microwave energy to tissue.

According to an aspect of the above-described embodiment, the method includes verifying a position of the ablation device within the surgical site based on the representation of the active heating zone. The method may include adjusting a position of the ablation device within the surgical site based on the verified position of the ablation device. The method may include adjusting the delivery of electrosurgical energy to the ablation device based on the verified position of the ablation device. The method may include continuing the delivery of electrosurgical energy to the target tissue via the ablation device to complete ablation of the target tissue.

According to an aspect of the above-described embodiment, the representation of the active heating zone is generated on a display in 3D.

According to an aspect of the above-described embodiment, the method may include adjusting the delivery of electrosurgical energy to the target tissue based on the representation of the active heating zone. The representation of the active heating zone may be based on at least one of a size, a surface area, a geometry, a shape, or a location of the active heating zone generated by the delivery of electrosurgical energy to the target tissue. The representation of the active heating zone may include a color coded gradient.

According to an aspect of the above-described embodiment, the method may include detecting a gap in the imaging data. The method may include manipulating the imaging device to optimize the representation of the active heating zone based on the detected gap in the imaging data. The representation of the active heating zone may be based on at least one of the electrosurgical energy delivered to the target tissue by the ablation device or a type of target tissue.

According to an aspect of the above-described embodiment, delivering electrosurgical energy to the target tissue may include pulsing the delivery of electrosurgical energy. Generating the representation o the active heating zone may include distinguishing Doppler shifts related to the active heating zone from Doppler shifts related to physiology based on a Doppler shift phase change differential.

According to another embodiment of the present disclosure, a system of generating a representation of an active heating zone on a display in real time during an ablation procedure includes a display, an ultrasound probe, an electrosurgical generator, an ablation device, and a processing unit. The ultrasound probe is configured to generate imaging data of a surgical site. The electrosurgical generator is configured to supply electrosurgical energy. The ablation device is coupled to the electrosurgical generator and is configured to deliver electrosurgical energy to target tissue within the surgical site. The processing unit is coupled to the display and is configured to process imaging data generated by the ultrasound probe to detect a Doppler shift in the imaging data. The Doppler shift is based on the delivery of electrosurgical energy to the target tissue. The process unit generates a representation of an active heating zone on the display overlaid on an image of the surgical site based on the detected Doppler shift. The representation of the active heating zone is based on the delivery of electrosurgical energy to the target tissue via the ablation device.

According to an aspect of the above-described embodiment, the system includes a visualization control in communication with the processing unit. The visualization control is configured to selectively generate the representation of the active heating zone on the display. The visualization control may be disposed on at least one of the display, the ablation device, or the ultrasound probe.

According to an aspect of the above-described embodiment, the representation of the active heating zone is a graphical representation of an active heating zone generated by the delivery of the electrosurgical energy to the target tissue via the ablation device. The system may include a field generator that is configured to track a location of at least one of the ablation probe or the ultrasound probe.

Further, to the extent consistent with this specification, any of the aspects described herein may be used in conjunction with any or all of the other aspects described herein.

### BRIEF DESCRIPTION OF THE DRAWINGS

Various aspects of the present disclosure are described hereinbelow with reference to the drawings, which are incorporated in and constitute a part of this specification, wherein:

FIG. 1 is a schematic diagram of a tissue ablation system in accordance with an embodiment of the present disclosure;

FIG. 2 is a schematic diagram of a processing unit of the ablation system of FIG. 1 in accordance with an embodiment of the present disclosure;

FIG. 3 is an enlarged view of a display of the ablation system of FIG. 1;

FIG. 4 is a schematic diagram of an ablation probe and an ultrasound probe of the ablation system of FIG. 1 placed relative to a surgical site; and

FIG. 5 is a flowchart of a method of generating a representation of an active heating zone on a display in real time during an ablation procedure in accordance with the present disclosure.

### DETAILED DESCRIPTION

The present disclosure provides for a method of generating a representation of an active heating zone on a display in real time during an ablation procedure. More specifically, as electrosurgical energy is delivered to target tissue during a tissue ablation procedure, the target tissue increases in temperature. The area of tissue increasing in temperature as a result of application of electrosurgical energy from an ablation device may be referred to as an “active heating zone.” The present disclosure provides for a system configured to process imaging data generated via ultrasound imaging to detect Doppler shifts in the imaging data, as detailed below. Based on the detection of Doppler shifts, an active heating zone is identified by the system and a representation of the active heating zone is generated and overlaid on an image of a surrounding surgical site on a suitable display. The representation of the active heating zone overlaid on the image of the surgical site provides the clinician with a visualization of where in the surgical site tissue is being ablated.

As it is used in this description, “microwave” generally refers to electromagnetic waves in the frequency range of 300 megahertz (MHz) (3×10⁸ cycles/second) to 300 gigahertz (GHz) (3×10¹¹ cycles/second). As it is used in this description, “ablation procedure” generally refers to any tissue ablation procedure, such as, for example, microwave ablation, radiofrequency (RF) ablation, or microwave or RF ablation-assisted resection. As it is used in this description, “transmission line” generally refers to any transmission medium that can be used for the propagation of signals from one point to another.

Embodiments of the present disclosure are now described in detail with reference to the drawings in which like reference numerals designate identical or corresponding elements in each of the several views. As used herein, the term “clinician” refers to a doctor, a nurse, or any other care provider and may include support personnel.

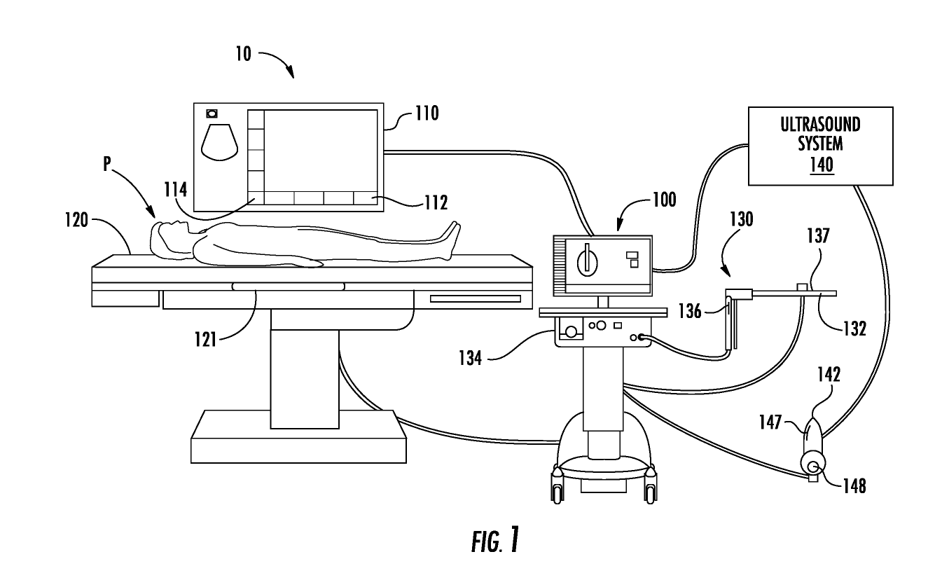

Referring now to FIG. 1, an ablation system 10 includes a processing unit 100, a display 110, a table 120, an ablation probe 130, and an ultrasound system 140. The processing unit 100 includes an electrosurgical generator 134 in electrical communication with the ablation probe 130. The generator 134 supplies electrosurgical energy to the ablation probe 130 for delivery of the electrosurgical energy to tissue. The ablation probe 130 may include a substantially rigid antenna 132 (e.g., a microwave antenna), as shown in the illustrated embodiments of FIGS. 1, 3, and 4, or may be in the form of a flexible ablation catheter. An exemplary ablation catheter is disclosed in U.S. Patent Publication No. 2015/0073407, the entire contents of which are hereby incorporated by reference.

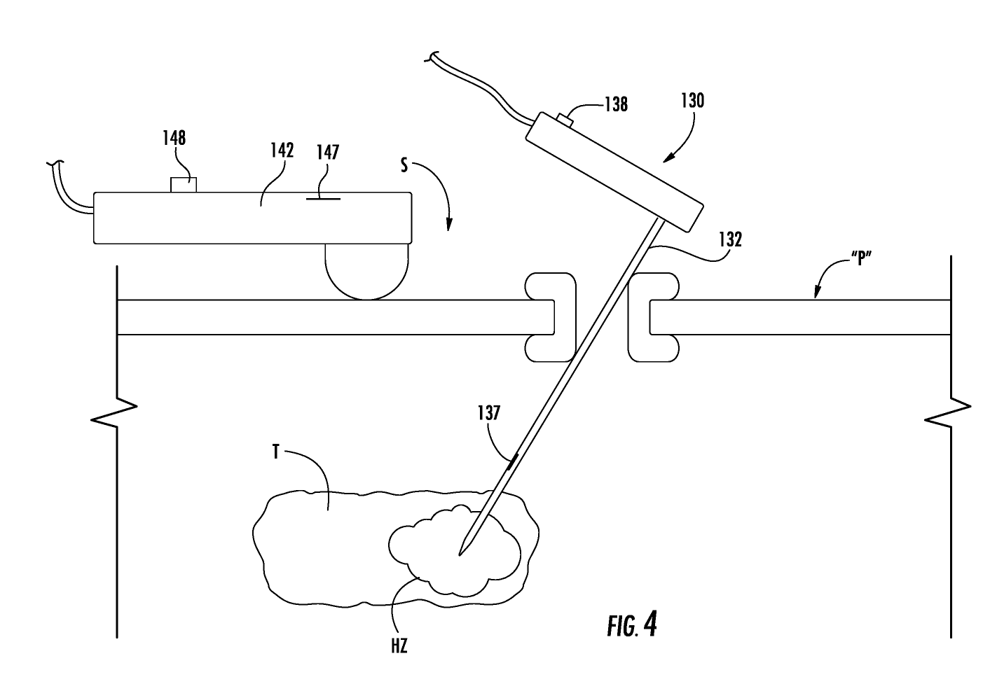

The ultrasound system 140 includes an ultrasound probe 142 and is in communication with the processing unit 100. The ultrasound probe 142 transmits ultrasound waves through a surgical site, referenced in FIG. 4 as surgical site “S”, and generates imaging data based on the reflection of the ultrasound waves from anatomical structures (e.g., tissue, bone, organs, etc.) back to the ultrasound probe 142. The imaging data is processed by the processing unit 100, which processes the imaging data to generate images of the surgical site “S” on the display 110 in real time. In some embodiments, the ultrasound system 140 may be integrated into the ablation system 10 as an ultrasound module that serves to provide imaging functionality before, during, and/or after ablation procedures (e.g., tumor detection, liver tissue disease staging, antenna tracking, zone monitoring, etc.).

With continued reference to FIG. 1, the table 120 supports a patient “P” during an ablation procedure. The table 120 may include a field generator 121 configured to track the location of the ablation probe 130 and/or the ultrasound probe 142 in real time. To that end, the ablation probe 130 and the ultrasound probe 142 may include markers 137, 147, respectively, that provide fiducial points of reference which may be detected by the field generator 121 and transmitted to the processing unit 100 to track the location of the ablation probe 130 and the ultrasound probe 142 during an ablation procedure. Additionally, prior to an ablation procedure, the ablation system 10 may be registered to the surgical site “S” of the patient “P” such that the location of the ablation probe 130 and/or the ultrasound probe 142 relative to structures within the surgical site “S” is trackable in real time by the processing unit 100. For a detailed description of an exemplary field generator and method of device position tracking, reference may be made to U.S. Provisional Patent Application No. 62/154,924, filed Apr. 30, 2015, entitled “METHODS FOR MICROWAVE ABLATION PLANNING AND PROCEDURE,” the entire contents of which are hereby incorporated by reference.

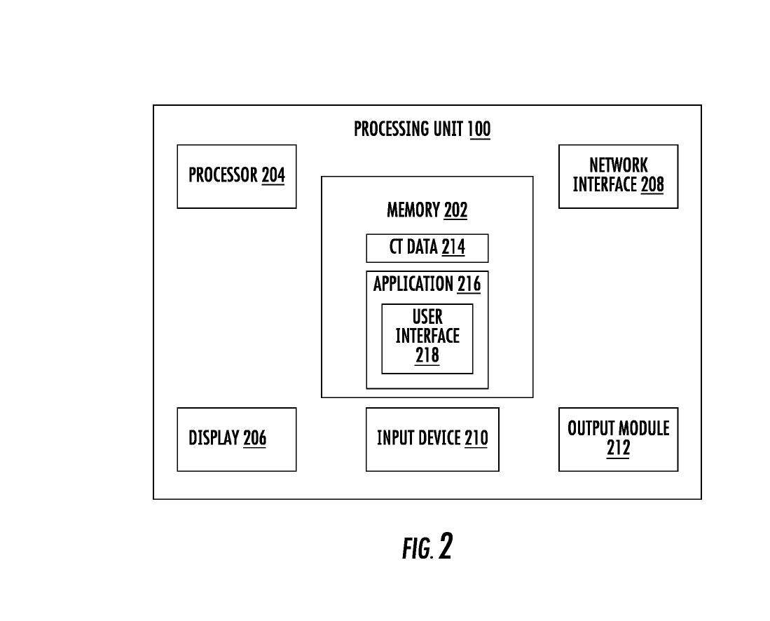

Turning now to FIG. 2, a system diagram of the processing unit 100 is shown in accordance with an embodiment of the present disclosure. The processing unit 100 may include memory 202, a processor 204, a display 206, a network interface 208, an input device 210, and an output module 212.

The memory 202 includes any non-transitory computer-readable storage media for storing data and/or software that is executable by the processor 204 and which controls the operation of the processing unit 100. In an embodiment, the memory 202 may include one or more solid-state storage devices such as flash memory chips. Alternatively or in addition to the one or more solid-state storage devices, the memory 202 may include one or more mass storage devices connected to the processor 204 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 204. That is, computer readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, Blu-Ray or other optical storage, magnetic cassettes, magnetic tape, magnetic disk storage or other magnetic storage devices, or any other medium which can be used to store the desired information and which can be accessed by the processing unit 100.

The memory 202 may store an application 216 and/or CT data 214. The application 216 may, when executed by the processor 204, cause the display 206 to present the user interface 218.

The processor 204 may be a general purpose processor, a specialized graphics processing unit (GPU) configured to perform specific graphics processing tasks while freeing up the general purpose processor to perform other tasks, and/or any number or combination of such processors.

The display 206 may be touch sensitive and/or voice activated, enabling the display 206 to serve as both an input and output device. Alternatively, a keyboard (not shown), mouse (not shown), or other data input devices may be employed. The display 206 is configured to operate in conjunction with the processing unit 100 to provide the clinician with the ability to control the generator 134 and/or navigation of the ablation probe 130.

The network interface 208 may be configured to connect to a network such as a local area network (LAN) consisting of a wired network and/or a wireless network, a wide area network (WAN), a wireless mobile network, a Bluetooth network, and/or the internet. For example, the processing unit 100 may receive computed tomographic (CT) image data of a patient from a server, for example, a hospital server, internet server, or other similar servers, for use during surgical ablation planning. Patient CT image data may also be provided to the processing unit 100 via a removable memory 202. The processing unit 100 may receive updates to its software, for example, the application 216, via the network interface 208. The processing unit 100 may also display notifications on the display 206 that a software update is available.

The input device 210 may be any device by means of which a user may interact with the processing unit 100, such as, for example, a mouse, keyboard, foot pedal, touch screen, and/or voice interface.

The output module 212 may include any connectivity port or bus, such as, for example, parallel ports, serial ports, universal serial busses (USB), or any other similar connectivity port known to those skilled in the art.

The application 216 may be one or more software programs stored in the memory 202 and executed by the processor 204 of the processing unit 100. During the planning phase, the application 216 guides a clinician through a series of steps to identify a target, size the target, size a treatment zone, and/or determine an access route to the target for later use during the procedure phase. In some embodiments, the application 216 is loaded on computing devices in an operating room or other facility where surgical procedures are performed, and is used as a plan or map to guide a clinician performing a surgical procedure, but without any feedback from the ablation probe 130 used in the procedure to indicate where ablation probe 130 is located in relation to the plan. In other embodiments, the ablation system 10 provides the processing unit 100 with data regarding the location of the ablation probe 130 within the body of the patient, such as by electromagnetic tracking, which the application 216 may then use to indicate on the plan where the ablation probe 130 and/or the ultrasound probe 142 is located.

The application 216 may be installed directly on the processing unit 100, or may be installed on another computer, for example a central server, and opened on the processing unit 100 via the network interface 208. The application 216 may run natively on the processing unit 100, as a web-based application, or any other format known to those skilled in the art. In some embodiments, the application 216 will be a single software program having all of the features and functionality described in the present disclosure. In other embodiments, the application 216 may be two or more distinct software programs providing various parts of these features and functionality. For example, the application 216 may include one software program for use during the planning phase, and a second software program for use during the procedure phase of the microwave ablation treatment. In such instances, the various software programs forming part of the application 216 may be enabled to communicate with each other and/or import and export various settings and parameters relating to the ablation treatment and/or the patient to share information. For example, a treatment plan and any of its components generated by one software program during the planning phase may be stored and exported to be used by a second software program during the procedure phase.

The application 216 communicates with a user interface 218 that presents visual interactive features to a clinician, for example, on the display 206 and that receives clinician input, for example, via a user input device. The user interface 218 may generate a graphical user interface (GUI) and output the GUI to the display 206 for viewing by a clinician.

The processing unit 100 is linked to the display 110, thus enabling the processing unit 100 to control the output on the display 110 along with the output on the display 206. The processing unit 100 may control the display 110 to display output which is the same as or similar to the output displayed on the display 206. For example, the output on the display 206 may be mirrored on the display 110. Alternatively, the processing unit 100 may control the display 110 to display different output from that displayed on the display 206. For example, the display 110 may be controlled to display guidance images and information during an ablation procedure, while the display 206 is controlled to display other output, such as configuration or status information.

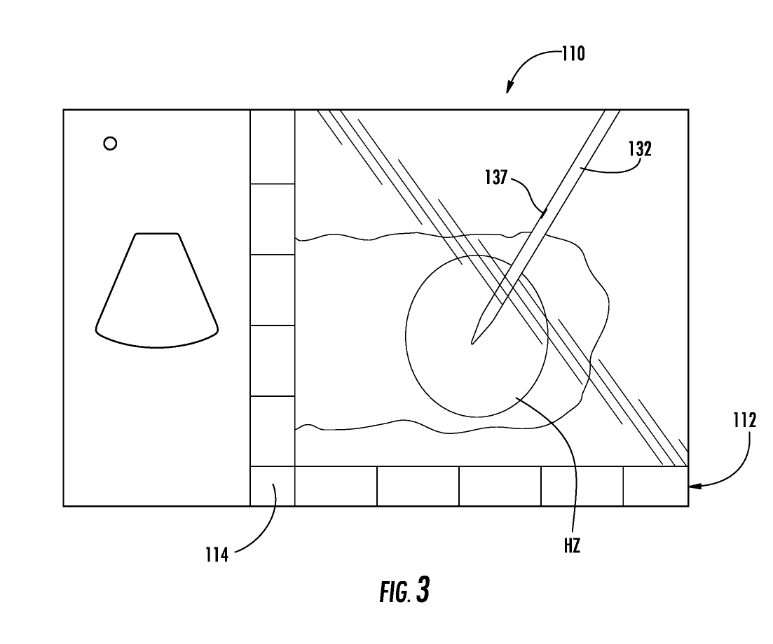

Generally, with reference to FIGS. 3 and 4, tissue undergoing an ablation procedure increases in temperature as ablation energy is applied to the tissue (e.g., via the ablation probe 130). Upon termination of the application of ablation energy, the tissue decreases in temperature. As ultrasound waves generated by the ultrasound probe 142 encounter the active heating zone, ultrasound wavelength may be Doppler shifted by heating-related mechanical agitations of the actively heated tissue. Additionally, as the clinician moves, rotates, or manipulates the ultrasound probe 142 relative to the patient such that the ultrasound waves generated by the ultrasound probe 142 change from passing through the active heating zone to passing through surrounding tissue “T”, or vice-versa, the degree of Doppler shift (as measured by intensity or phase changes per unit time) of the ultrasound waves may be caused by a difference in intensity of the active heating profile between the various tissues surrounding the ablation probe 130 and the surrounding tissue “T”. This mechanical shift of the ultrasound wavelength is known as a “Doppler shift.”

Heating-related ultrasound wavelength shifts occur due to mechanical agitation of the tissue from the active heating induced temperature rise. Contributing to the mechanical agitation are factors including, but not limited to, fluid movement through tissue due to pressure changes across the heated tissue, anatomical material phase change at temperature thresholds, and electromagnetic forces acting upon tissue at a molecular level. Heating-related Doppler shifts are distinct from the Doppler shifts used to image blood flowing through a vessel or within the heart, or to detect bleeding. Blood flow related Doppler shifts have a more consistent phase (red or blue shift) across a larger area than do heating-related Doppler shifts, due to a volume of fluid moving together through the body in a confined lumen or chamber with generally consistent direction. With blood flow, the volume of fluid also generally moves together at a similar velocity, accelerating and decelerating at a rate consistent with cardiac, arterial, or vascular flow behavior. With heat-related Doppler shift, the phase of the Doppler shift (red or blue) may differ across very small distances. Also, the magnitude of the Doppler shift exhibits a high differential in time due to rapid phase shift between red and blue.

As the ultrasound system 140 (FIG. 1) receives imaging data from the ultrasound probe 142, the ultrasound system 140 transmits the imaging data to the processing unit 100 in real time during an ablation procedure. The processing unit 100 (FIG. 1) is configured to detect the unique characteristics of heating-related Doppler shifts received from the ultrasound system 140. Based on these detected Doppler shifts, the active heating zone is identified by the processing unit 100 and displayed as a graphical representation of the active heating zone (referenced in FIGS. 3 and 4 as an active heating zone “HZ”) on the display 110 overlaid on the image of the surgical site “S”. The representation of the active heating zone “HZ” may be based on any one or more characteristics of the active heating zone “HZ” including, but not limited to, size, surface area, geometry, shape, and/or location determined by the processing unit 100 upon processing the imaging data received from the ultrasound system 140. The representation of the active heating zone “HZ” may be displayed in two dimensions (“2D”) or three dimensions (“3D”). In some embodiments, the representation of the active heating zone “HZ” may be depicted as a generally circular shape, as shown in FIG. 3. In some embodiments, the representation of the active heating zone “HZ” may be depicted as a generally cloud-like shape, as shown in FIG. 4. This representation of the active heating zone “HZ” overlaid on the surgical site “S” on the display 110 provides the clinician with a visualization of where in the surgical site “S” tissue is being ablated. In generating the representation of the active heating zone “HZ”, the processing unit 100 may take into consideration parameters such as, but not limited to, the power of the electrosurgical energy being delivered from the generator 134 and the type of tissue (e.g., bone, lung, liver, etc.) being ablated.

In some embodiments, display of the active heating zone “HZ” may include an intensity map related to Doppler shift parameters. This display may display boundaries across the active heating zone “HZ” that correspond to values for given Doppler shift parameters that exceed specific thresholds to demonstrate clinically relevant levels of active heating. Boundary groupings may be used as clinical outcome prediction zones and may indicate active heating intensity that may result in significant desiccation of the tissue after a given activation time, far exceeding toxic heating levels, thereby indicating to the user that the procedure will result in potentially undesirable levels of heating. Other boundary groupings may indicate active heating intensity that is below a particular level, which assures the procedure will reach toxic heating levels, thereby indicating to the user that the energy should be increased or the device should be replaced to gain adequate ablation coverage. These boundaries provide real time feedback to the user to optimize the heating effect spatially within areas of targeted tissue surrounded by healthy tissue or critical anti-targeted anatomical structures. Doppler shift parameters may include the peak intensity of the redshift and blueshift observed on average or instantaneously at a given spatial location within tissue, the number of phase changes between redshift to blueshift at a given spatial location within tissue over a given integration time, the differential in Doppler shift magnitude, or other metrics related to the active heating zone generated Doppler shifts. The value of these Doppler shift parameters may be determined by the system using lookup tables or user-customized thresholds and displayed to the user with spatial context to the patient, tracked surgical tools, and/or natural and artificial fiducials.

The processing unit 100 may also pulse with on and off sequence the electrosurgical energy delivered to the ablation probe 130 to enable further separation between Doppler shift caused by anatomical processes (breathing, beating heart, blood flow, digestive movement, etc.) from Doppler shifts caused by the active heating from the ablation probe 130. During delivery of electrosurgical energy to the ablation probe 130, the total Doppler shift observed across the imaging space would include both the active heating and anatomical Doppler shift contributions. When the delivery of electrosurgical energy to the ablation probe 130 is terminated, the residual Doppler shift observed across the imaging space would be from anatomical processes. An additional decaying component to the active heating Doppler shift immediately after energy delivery is terminated may be detected by the processor unit 100 to determine spatially thermal mass and temperature. In addition, the processing unit 100 may detect anatomical structures within the surgical site “S” based on the imaging data and display these structures on the display 110 relative to the surgical site “S” and the active heating zone “HZ”. The processing unit 100 may separate active heating zone associated Doppler shifts from non-heating associated Doppler shifts (blood flowing through vessels, beating heart, breathing motions, bowl motions, etc.) and clearly distinguish these observations on the system visualization tools via the display 110.

Ultrasound waves generated by the ultrasound probe 142 may undergo a positive Doppler phase shift (e.g., a phase shift moving away from the ultrasound probe 142) or a negative Doppler phase shift (e.g., a phase shift moving towards the ultrasound probe 142). In some embodiments, a positive Doppler phase shift may be represented by a first color (e.g., red) on the display 110 and a negative Doppler phase shift may be represented by a second color (e.g., blue) on the display 110. In this way, the representation of the active heating zone “HZ” on the display 110 may be color coded to depict positive and negative Doppler phase shifts.

In some embodiments, the representation of the active heating zone “HZ” on the display 110 may include color coded temperature gradients that are indicative of differences in potential temperature rise across various locations of the active heating zone generated by the ablation probe 130, the differential in potential temperature rise with time within the active heating zone generated by the ablation probe 130, and/or at what time rate of change the temperature is changing at particular locations of the active heating zone generated by the ablation probe 130.

The processing unit 100 is configured to recognize gaps in the imaging data received from the ultrasound system 140. In response to the detected gaps in the imaging data, the processing unit 100 may provide feedback to the clinician to sweep, rotate, pivot, move, or otherwise manipulate the ultrasound probe 142 to eliminate the gaps thereby creating a complete visualization of the surgical site “S” on the display 110. For example, the processing unit 100 may provide visual feedback on the display 110, audible feedback, and/or haptic feedback via vibration of the ultrasound probe 142 or the ablation antenna 132. Gaps in the imaging data may be classified by the processing unit 100 as areas in the imaging space that do not exhibit either anatomical structures, active heating, or the ablation probe 130. Potential causes of gaps in the imaging data may include, but are not limited to, gas, bones, or lungs within the imaging field of view. The processing unit 100 may specifically recognize the bubble field generated by the phase change in anatomical fluids within the ablated zone.

With reference to FIG. 3, the display 110 includes a user interface 112 that functions as a touch screen to receive inputs from a clinician. The user interface 112 may include buttons, toggles, switches, keyboards, or other known types of interfaces for accepting input from the clinician. In some embodiments, the user interface 112 may include a heating zone visualization control 114 that enables a clinician to generate a representation of the active heating zone “HZ” overlaid on the surgical site “S” in real time during an ablation procedure. Additionally or alternatively, the ultrasound probe 142 may include a visualization control 148 and/or the ablation probe 130 may include a visualization control 138. Each of the visualization controls 114, 138, and 148 are in communication with the processing unit 100 such that when activated, the processing unit 100 generates the representation of the active heating zone “HZ” on the display 110 for visualization by a clinician in real time during an ablation procedure.

In some embodiments, the ultrasound system 140 is a 3D ultrasound system which enables three-dimensional imaging of the surgical site “S” and for generating three-dimensional representations of the active heating zone “HZ” on the display 110. To capture a three-dimensional image of the surgical site “S”, the ultrasound probe 142 may be swept across the surgical site “S” and/or be rocked on the patient to generate and capture real time 3D imaging data of the surgical site “S”. Additionally or alternatively, the ultrasound system 140 may include a second ultrasound probe (not shown) that cooperates with the ultrasound probe 142 to capture 3D imaging data of the surgical site “S”. In such embodiments, the processing unit 100 analyzes the 3D imaging data to display 3D images of the surgical site “S” on the display 110 in real time.

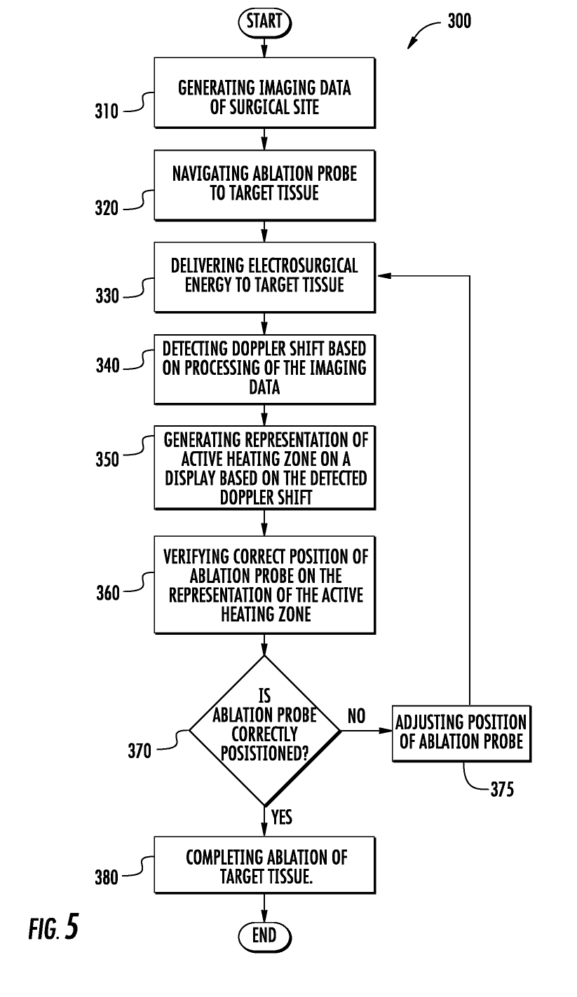

Referring now to FIG. 5, a method 300 of generating a representation of an active heating zone on a display in real time during an ablation procedure is described in accordance with the present disclosure. For purposes of illustration, the method 300 is described below with respect to the ablation system 10 detailed above with reference to FIGS. 1-4.

Initially, at step 310, the clinician uses the ultrasound probe 142 to generate imaging data of the surgical site “S”. The imaging data is received from the ultrasound system 140 by the processing unit 100, which processes the imaging data and generates images of the surgical site “S” on the display 110 in real time.

At step 320, the clinician navigates the ablation probe 130 relative to the surgical site “S” such that the ablation probe 130 is positioned in suitable proximity to target tissue. An image or images of the surgical site “S” may be registered and loaded into the memory 202 of the processing unit 100 of the ablation system 10 to aid a clinician in positioning the ablation probe 130 in proximity to the target tissue while viewing the surgical site “S” on the display 110. An exemplary method of positioning an ablation probe relative to tissue is disclosed in U.S. Patent Publication No. 2016/0000302, the entire contents of which are hereby incorporated by reference. The images of the surgical site “S” on the display 110 may be generated based on imaging data received from the ultrasound system 140 and processed by the processing unit 100. In some embodiments, images of the surgical site “S” may be actual images captured by cameras (not shown) or other surgical imaging systems (e.g., ultrasound, x-ray, etc.) or the images may be graphical representations of the surgical site “S” generated via the registration and real time tracking of surgical instruments (e.g., ablation probe 130 and ultrasound probe 142). In some embodiments, the clinician may navigate the ablation probe 130 to target tissue utilizing the ultrasound system 140. Navigation instructions, such as a pathway and other relevant information, may be displayed on the display 110.

When the ablation probe 130 is positioned in suitable proximity to the target tissue, the clinician, at step 330, delivers electrosurgical energy to the target tissue via the ablation probe 130 to generate an active heating zone.

At step 340, the processing unit 100 detects Doppler shifts in the imaging data received from the ultrasound system 140 and, at step 350, the representation of the active heating zone “HZ” is generated by the processing unit 100 and overlaid on the image of the surgical site “S”. In some embodiments, the representation of the active heating zone “HZ” is generated automatically by the processing unit 100 and visualized on the display 110 in real time during an ablation procedure. In some embodiments, the clinician activates one of the visualization controls 114, 138, 148 on the user interface 112 of the display 110, the ablation probe 130, or the ultrasound probe 142 to generate the representation of the active heating zone “HZ” overlaid on the image of the surgical site “S”. The representation of the active heating zone “HZ” is generated by the processing unit 100 based on the detected Doppler shifts and enables the clinician to visualize where tissue is being ablated and, thus, whether the ablation probe 130 is correctly positioned within the surgical site “S” to effect ablation of the target tissue.

At step 360, based on the generated representation of the active heating zone “HZ” on the display 110, the clinician may verify, in real time during the ablation procedure, that the ablation probe 130 is correctly positioned within the surgical site “S”. At step 370, if the clinician determines that the ablation probe 130 is not correctly positioned, the clinician may adjust the position of the ablation probe 130 at step 375 and return to step 330 to continue or re-initialize delivery of electrosurgical energy to the target tissue. In some embodiments, the clinician may choose to adjust (e.g., decrease or terminate) the delivery of electrosurgical energy to the ablation probe 130 until the clinician is able to verify that the ablation probe 130 is correctly positioned relative to the target tissue through visual confirmation via the representation of the active heating zone “HZ” on the display 110.

At step 370, if the clinician determines that the ablation probe 130 is correctly positioned, the clinician continues to deliver electrosurgical energy to the target tissue via the ablation probe until ablation is complete (e.g., an ablation lesion has been formed in the target tissue).

In some embodiments, the processing unit 100 is capable of determining, based on processing of imaging data received from the ultrasound system 140, if the ultrasound probe 142 is not optimally positioned relative to the surgical site “S” for generating an optimum representation of the active heating zone “HZ”. For example, if there is a lack of detection of Doppler shifts in the imaging data received from the ultrasound system 140, the processing unit 100 may provide visual feedback on the display 110 to instruct the clinician to move, sweep, or pivot the ultrasound probe 142 to optimize the representation of the active heating zone “HZ”. Additionally or alternatively, the processing unit 100 may provide visual, haptic, or audible feedback via the ablation probe 130 and/or the ultrasound probe 142 to move, sweep, or pivot the ultrasound probe 142 to optimize the representation of the active heating zone “HZ” on the display 110.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

What is claimed is:

1. A method of generating a representation of an active heating zone on a display in real time during an ablation procedure, the method comprising:
processing imaging data of a surgical site generated by an imaging device;
navigating an ablation device in proximity to target tissue within the surgical site;
delivering electrosurgical energy to the target tissue via the ablation device to generate an active heating zone;
detecting a Doppler shift in the imaging data based on the delivery of electrosurgical energy to the target tissue; and
generating a representation of the active heating zone relative to the surgical site based on the detected Doppler shift.

2. The method according to claim 1, wherein the representation of the active heating zone is a graphical representation of the active heating zone generated on a display by a processing unit coupled to the display and configured to process the imaging data generated by the imaging device.

3. The method according to claim 1, wherein generating the representation of the active heating zone includes indicating a clinical outcome prediction zone based on active heating intensity.

4. The method according to claim 1, further comprising verifying a position of the ablation device within the surgical site based on the representation of the active heating zone.

5. The method according to claim 4, further comprising adjusting a position of the ablation device within the surgical site based on the verified position of the ablation device.

6. The method according to claim 4, further comprising adjusting the delivery of electrosurgical energy to the ablation device based on the verified position of the ablation device.

7. The method according to claim 4, further comprising continuing the delivery of electrosurgical energy to the target tissue via the ablation device to complete ablation of the target tissue.

8. The method according to claim 1, wherein the representation of the active heating zone is generated on a display in 3D.

9. The method according to claim 1, further comprising adjusting the delivery of electrosurgical energy to the target tissue based on the representation of the active heating zone.

10. The method according to claim 1, wherein the representation of the active heating zone is based on at least one of a size, a surface area, a geometry, a shape, or a location of the active heating zone generated by the delivery of electrosurgical energy to the target tissue.

11. The method according to claim 1, wherein the representation of the active heating zone includes a color coded gradient.

12. The method according to claim 1, further comprising detecting a gap in the imaging data.

13. The method according to claim 12, further comprising manipulating the imaging device to optimize the representation of the active heating zone based on the detected gap in the imaging data.

14. The method according to claim 1, wherein delivering electrosurgical energy to the target tissue includes pulsing the delivery of electrosurgical energy.

15. The method according to claim 1, wherein generating the representation of the active heating zone includes distinguishing Doppler shifts related to the active heating zone from Doppler shifts related to physiology based on a Doppler shift phase change differential.

16. A system for generating a representation of an active heating zone on a display in real time during an ablation procedure, the system comprising:
a display;
an ultrasound probe configured to generate imaging data of a surgical site;
an electrosurgical generator configured to supply electrosurgical energy;
an ablation device coupled to the electrosurgical generator and configured to deliver electrosurgical energy to target tissue within the surgical site; and
a processing unit coupled to the display and configured to process imaging data generated by the ultrasound probe to detect a Doppler shift in the imaging data, the Doppler shift based on the delivery of electrosurgical energy to the target tissue,
wherein the processing unit generates a representation of an active heating zone on the display overlaid on an image of the surgical site based on the detected Doppler shift, the representation of the active heating zone based on the delivery of electrosurgical energy to the target tissue via the ablation device.

17. The system according to claim 16, further comprising a visualization control in communication with the processing unit and configured to selectively generate the representation of the active heating zone on the display.

18. The system according to claim 17, wherein the visualization control is disposed on at least one of the display, the ablation device, or the ultrasound probe.

19. The system according to claim 16, wherein the representation of the active heating zone is a graphical representation of an active heating zone generated by the delivery of the electrosurgical energy to the target tissue via the ablation device.

20. The system according to claim 16, further comprising a field generator configured to track a location of at least one of the ablation probe or the ultrasound probe.
